(19) United States
(12) Patent Application Publication
(10) Pub. No.: US 2023/0386624 A1
(43) Pub. Date: Nov. 30, 2023

(54) DATA PROCESSING APPARATUS AND METHOD

(71) Applicant: CANON MEDICAL SYSTEMS CORPORATION, Otawara-shi (JP)

(72) Inventors: David Innes MILLER, Edinburgh (GB); Yvonne BELTON, Edinburgh (GB)

(73) Assignee: CANON MEDICAL SYSTEMS CORPORATION, Otawara-shi (JP)

(21) Appl. No.: 17/664,977

(22) Filed: May 25, 2022

(51) Int. Cl. G16H 10/60

(52) U.S. Cl. CPC G16H 10/60

(57) ABSTRACT

A clinical information system comprises processing circuitry configured to: receive a user input from a user, wherein the user input instructs the performing of a first action on first medical data for a subject; determine based on the user input and/or the first action at least one input term; determine at least one further term that is conceptually related to the at least one input term; determine whether any stored action of a set of stored actions is associated with the at least one further term; and if a stored action is associated with the at least one further term: perform said stored action on second medical data for the subject; and provide to the user a notification of said stored action.

### FIELD

Embodiments described herein relate generally to a data processing apparatus and method, for example an apparatus and method for processing clinical data relating to a patient or other subject.

### BACKGROUND

The amount of data captured for a given patient by hospital systems is ever increasing. Medical records may contain large quantities of data even for a single patient.

An Electronic Medical Record relating to a patient may contain both unstructured data and structured data. The unstructured data may comprise, for example, clinical notes that comprise free text provided by clinicians. The structured data may comprise vital sign measurement results which may include, for example, measurements of temperature, blood pressure, pulse rate and/or respiration rate. The structured data may comprise laboratory results which may include, for example, the results of blood or urine tests. The structured data may comprise imaging data. The structured data may comprise medication data.

It is known for a clinical user, for example a physician, to perform various tasks with relation to stored medical data. For example, the clinical user may enter a search term into a system that highlights all instances of the search term in an unstructured text document. The clinical user may run an algorithm to analyze structured data, for example to find all instances of blood pressure measurements that exceed a certain value. Various different tasks may be performed.

Clinical users may typically be overloaded with information and may find it difficult to find key information for a current clinical decision. Clinical users may be very busy and may have little time to actively search out key information.

Automated systems may struggle to understand a current clinical context and so may struggle to know what information is useful to highlight to the user now, versus what information would merely provide a distraction to the user.

One option may be to speculatively run automated systems on all data. For example, when an Electronic Medical Record is opened, all available tasks may be performed on the Electronic Medical Record. However, speculatively running automated systems on all data can be costly in terms of hardware.

Rules may be applied to structured data. A rule may comprise an algorithm that generates findings for a user when run against structured data. A rule may be written by a clinical user. In some circumstances, pre-set rules may be provided and may be enabled or disabled by a clinical user.

A rule may be run against each available datum of a set of structured data. For each datum of structured data the rule may either pass or fail. For example a high blood pressure rule may be run on blood pressure data. For a datum when the blood pressure is greater than 140/90, the rule results in a pass. For a datum when the blood pressure is less than 140/90, the rule results in a fail. Some rules may use more complex concepts, for example moving averages. When a datum passes a rule then the user may be notified. The datum may be highlighted when visualized.

A text-expanding semantic search function may be integrated into a clinical notes panel. The clinical notes panel may comprise a screen or window that is displayed to the user. The clinical notes panel may display clinical notes that comprise unstructured text data.

The text-expanding semantic search function receives as an input a search term. When the text-expanding semantic search function receives the search term, it produces a set of terms that are related to the search term. The text-expanding semantic search function then finds related terms in a body of text, for example clinical notes, and returns a list of search findings comprising all instances of the related terms in the body of text. The clinical notes panel may then highlight each search finding to the clinical user.

In an example of a text-expanding semantic search, the user enters as an input the term ‘edema’. The text-expanding semantic search function determines a list of terms that are related to edema, which includes the terms ‘hypertension’, ‘hydrochlorothiazide’, ‘chest pain’, ‘shortness of breath’, ‘swelling’, ‘dizziness’, ‘extremities’, ‘facial’ and ‘vascular’. The list of related terms also includes the original search term ‘edema’. The text-expanding semantic search function is used to identify and highlight each instance of any of the related terms in an unstructured text document, which may also be referred to as a free text document.

### BRIEF DESCRIPTION OF THE DRAWINGS

Embodiments are now described, by way of non-limiting example, and are illustrated in the following figures, in which:

FIG. 1 is a schematic illustration of an apparatus in accordance with an embodiment;

FIG. 2 is a flow chart illustrating in overview a method of an embodiment;

FIG. 3A illustrates an example of a clinical notes panel showing a notification in accordance with an embodiment;

FIG. 3B illustrates an example of a vital signs data in which an output of an algorithm is highlighted in accordance with an embodiment;

FIG. 4A illustrates an example of a clinical notes panel showing a notification in accordance with an embodiment;

FIG. 4B illustrates an example a vital signs data in which an output of an algorithm is highlighted in accordance with an embodiment;

FIG. 5 illustrates an example of a findings panel in accordance with an embodiment;

FIG. 6 illustrates an example of a findings panel with summaries in accordance with an embodiment;

FIG. 7 shows a list of rules with associated search terms and trigger terms; and

FIG. 8 is a flow chart illustrating in overview a method of an embodiment.

### DETAILED DESCRIPTION

Certain embodiments provide a clinical information system, comprising processing circuitry configured to: receive a user input from a user, wherein the user input instructs the performing of a first action on first medical data for a subject; determine based on the user input and/or the first action at least one input term; determine at least one further term that is conceptually related to the at least one input term; determine whether any stored action of a set of stored actions is associated with the at least one further term; and, if a stored action is associated with the at least one further term, perform said stored action on second medical data for the subject; and provide to the user a notification of said stored action.

Certain embodiments provide a method comprising: receiving a user input from a user, wherein the user input instructs the performing of a first action on first medical data for a subject; determining based on the user input at least one input term; determining at least one further term that is conceptually related to the at least one input term; determining whether any stored action of a set of stored actions is associated with the at least one further term; and, if a stored action is associated with the at least one further term, performing said stored action on second medical data for the subject; and providing to the user a notification of said stored action

A clinical information system, comprising processing circuitry configured to: receive a user input from a user, wherein the user input instructs the processing circuitry to perform a first action on first medical data for a subject, wherein the first action comprises a rule or algorithm; determine based on the user input at least one input term that is associated with the first action; determine at least one further term that is conceptually related to the at least one input term; determine whether instances of the at least one further term are present in second medical data for the subject, wherein the second medical data comprises text data; and, if instances of the at least one further term are present in the second medical data, provide to the user a notification of said instances.

Certain embodiments provide a method comprising: receiving a user input from a user, wherein the user input instructs the processing circuitry to perform a first action on first medical data for a subject, wherein the first action comprises a rule or algorithm; determining based on the user input at least one input term that is associated with the first action; determining at least one further term that is conceptually related to the at least one input term; determining whether instances of the at least one further term are present in second medical data for the subject, wherein the second medical data comprises text data; and, if instances of the at least one further term are present in the second medical data, providing to the user a notification of said instances.

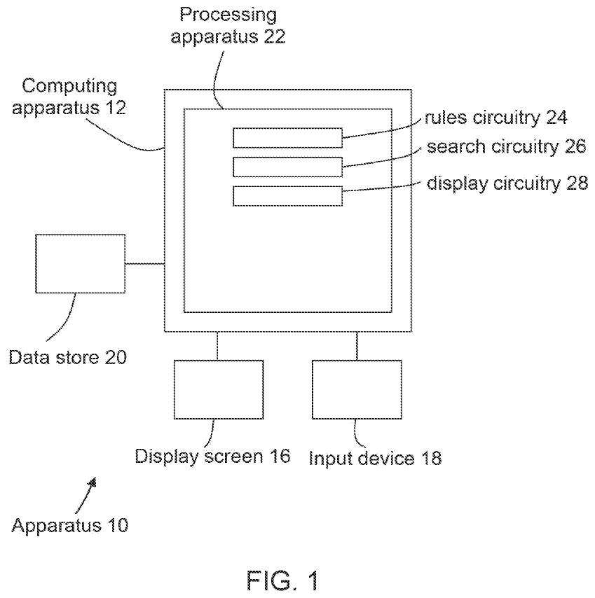

An apparatus 10 according to an embodiment is illustrated schematically in FIG. 1. The apparatus 10 may also be referred to as a clinical information system. In the present embodiment, the apparatus 10 is configured to process medical data, for example Electronic Medical Records. The medical data may comprise both structured data and unstructured data. For example, the structured data may comprise vital signs data and/or laboratory data and/or imaging data. The unstructured data may comprise free text data such as clinical notes.

In other embodiments, the apparatus 10 may be configured to process any appropriate data, which may comprise non-medical data.

The apparatus 10 comprises a computing apparatus 12, which in this case is a personal computer (PC) or workstation. The computing apparatus 12 is connected to a display screen 16 or other display device, and an input device or devices 18, such as a computer keyboard and mouse.

The computing apparatus 12 receives medical data from a data store 20. In alternative embodiments, computing apparatus 12 receives medical data from one or more further data stores (not shown) instead of or in addition to data store 20. For example, the computing apparatus 12 may receive medical data from one or more remote data stores (not shown) which may form part of an Electronic Medical Records system or Picture Archiving and Communication System (PACS).

Computing apparatus 12 provides a processing resource for automatically or semi-automatically processing medical text data. Computing apparatus 12 comprises a processing apparatus 22. The processing apparatus 22 comprises rules circuitry 24 configured to perform a plurality of rules, algorithms or other actions; search circuitry 26 configured to perform search functions which may include determining related terms and searching for the related terms; and display circuitry 28 configured to display information to a user, for example via display screen 16.

In the present embodiment, the circuitries 24, 26, 28 are each implemented in computing apparatus 12 by means of a computer program having computer-readable instructions that are executable to perform the method of the embodiment. However, in other embodiments, the various circuitries may be implemented as one or more ASICs (application specific integrated circuits) or FPGAs (field programmable gate arrays).

The computing apparatus 12 also includes a hard drive and other components of a PC including RAM, ROM, a data bus, an operating system including various device drivers, and hardware devices including a graphics card. Such components are not shown in FIG. 1 for clarity.

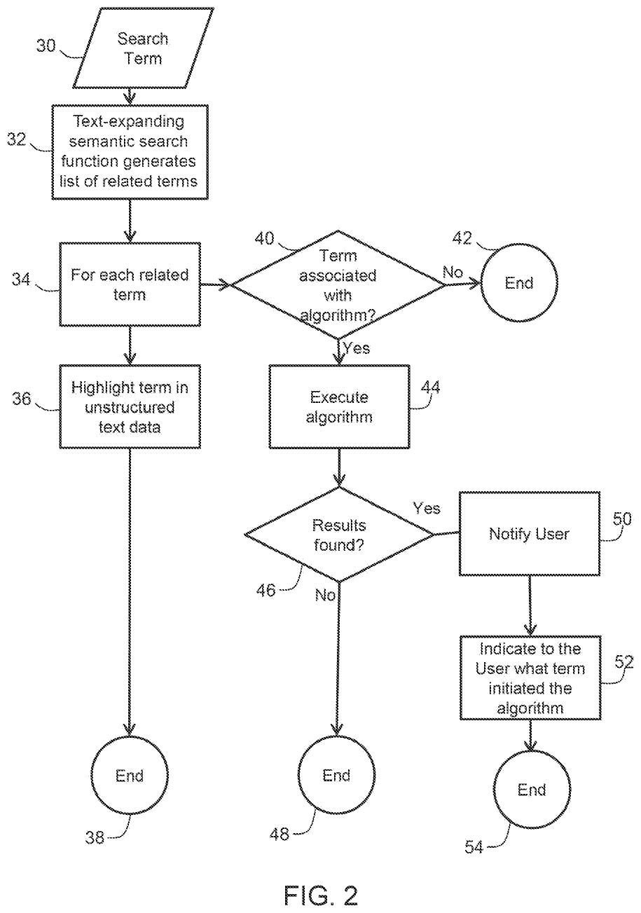

FIG. 2 is a flow chart illustrating in overview a method of an embodiment. The apparatus of FIG. 1 is configured to perform the method of FIG. 2.

The method of FIG. 2 is performed on a set of medical data relating to a patient. The set of medical data may be referred to as a corpus of medical data. The set of medical data may be an Electronic Medical Record. The set of medical data may be referred to as a patient record. In other embodiments, a corresponding method may be performed on medical data for any human or animal subject.

The set of medical data comprises both structured data and unstructured data. The structured data may comprise, for example, at least one of vital signs data, laboratory data, imaging data, medication data, observation data. The unstructured data comprises text data, for example clinical notes.

In other embodiments, the set of medical data may comprise any suitable medical data, for example one or more of a clinical note, a nursing note, a set of imaging data, a set of imaging measurements, a set of lab result data, a set of patient observation data, a set of vital sign data, a prescription, a medication record, data obtained from the patient, data obtained from a medical device, a summary report, a medical history report, a case conference report, a billing report, a radiology report, a set of patient events, medication data, administration or records data or any other suitable set of recorded information relating to the patient.

In the present embodiment the set of medical data is obtained from data store 20. In other embodiments the medical data may be obtained from any suitable data store or data stores, for example from multiple servers on a network. The medical data may be gathered from a healthcare informatics system or from a variety of healthcare informatics systems. The medical data may be formatted in any suitable electronic format, for example any known format for electronic medical record data. DICOM structured reports are one such possible format. In some embodiments, data pertaining to different types of medical data may have different data formats.

At stage 30 of FIG. 2, a clinical user, for example a physician, inputs a search term to conduct a search of first portion of the set of medical data. In other embodiments, the user may be any suitable user, for example any suitable medical professional or researcher.

In the present embodiment, the first portion of the set of medical data is a set of unstructured data comprising a set of clinical notes.

The user provides the search term as a user input to instruct a search for the search term to be performed. The user may type the search term into a search box or use any suitable input method.

The search circuitry 26 takes the search term to be an input term. The search term may comprise one or more characters. The search term may comprise a word, a partial word, or a group of words. In one example, the search term is ‘diabetes’. Further examples of search terms are described below.

At stage 32, the search circuitry 26 applies a text-expanding semantic search function to the input term to generate a plurality of related terms. The related terms are related conceptually to the search term. In the embodiment of FIG. 2, the related terms include the search term itself. The text-expanding semantic search function may also be described as a heuristic that can generate one or more related terms that are clinically relevant to the search term.

Each related term may also be described as a keyword. Each related term may comprise one or more characters. Each related term may comprise a word, a partial word, or a group of words.

The text-expanding semantic search function may determine related terms based at least in part on a clinical coding system. Clinical coding systems may also be known as terminologies or ontologies. Clinical coding systems express clinical concepts together with their relationships. Known clinical coding systems such as SNOMED CT (Systematized Nomenclature of Medicine—Clinical Terms), ICD (the International Statistical Classification of Diseases and Related Health Problems) and OCPS (OPCS Classification of Interventions and Procedures) are well-resourced and comprehensive. Clinical coding systems include clinical concepts and relationships between those concepts. Relationships between concepts may be represented as edges in a knowledge graph. The text-expanding semantic search function may determine a clinical concept associated with the search term and find related terms that are associated with the same clinical concept and/or related clinical concepts.

Additionally or alternatively, the text-expanding semantic search function may determine related terms based at least in part on at least one of: fuzzy matching using an edit distance such as a Levenshtein edit distance; phonetic matching using a matching algorithm, for example Metaphone; stemming; and/or dictionary look-up of abbreviations.

In other embodiments, the text-expanding semantic search function may use any suitable method to determine the related terms. A machine learning model may be used to determine the related terms, for example a model as described in U.S. patent application Ser. No. 17/011,363, which is hereby incorporated by reference.

In the example in which the search term is ‘diabetes’, the related terms may include various terms identified by the search procedure as being conceptually related to ‘diabetes’. For example, the related terms may include ‘Hypertension’, ‘High blood pressure’ and ‘High BP’.

At stage 34, the search circuitry 26 passes the related terms to the rules circuitry 24. The search circuitry 26 and rules circuitry 24 each commence processes for each of the related terms as described below with reference to stages 36 and 40.

Stage 36 is performed for each of the related terms. At stage 36, for each related term, the search circuitry 26 searches the clinical notes to identify each instance of the related term in the set of clinical notes.

The display circuitry 28 displays at least part of the set of clinical notes on a clinical notes panel. The display circuitry 28 highlights each instance of each related term in the set of clinical notes. For example, the display circuitry 28 may highlight each instance of a related term in the set of clinical notes using a coloured background region, an outline, a change of font or style, a flag, or any other suitable highlighting method.

Once each instance of each related term has been identified and highlighted in the clinical notes, the search process of stage 36 ends at stage 38.

At stage 40, for each of the related terms, the rules circuitry 24 determines whether any algorithms are associated with the related term.

A set of algorithms are stored in the apparatus 10, for example in the data store 20 or in any suitable data store. In the present embodiment, some of the stored algorithms of the set of stored algorithms have been written by the clinical user. Some of the stored algorithms of the set of stored algorithms are pre-set algorithms. Pre-set algorithms may be enabled or disabled by the clinical user.

Each stored algorithm has associated text, which may be entered by a user when a rule is created. The associated text comprises one or more clinical terms. For example, associated text for a high blood pressure rule may comprise the terms ‘high blood pressure’ and ‘hypertension’. The clinician may define the algorithm such that it is run whenever the term ‘High Blood Pressure’ is searched.

The associated text may be stored in combination with the stored algorithm, or may form part of the stored algorithm. The associated text relates to the subject matter of the algorithm and/or to circumstances under which the algorithm is to be performed.

In some circumstances, multiple stored algorithms may be associated with a single term. In some circumstances, multiple terms are associated with a single stored algorithm.

The rules circuitry 24 determines whether any of the stored algorithms are associated with each related term by searching the set of stored algorithms including their associated text. For example, the rules circuitry 24 may search a set of stored algorithms which may comprise a list, a table, a database, or any suitable format. In other embodiments, any suitable method of determining whether any actions are associated with each related term may be performed. For each related term, the rules circuitry 24 selects any stored algorithms in the set of stored algorithms that are associated with the related term.

In other embodiments, the rules circuitry 24 may determine whether any of the stored algorithms are associated with each related term in any suitable manner. For example, the rules circuitry 24 may determine whether algorithms are associated with related terms based on a type of data that each algorithm operates on in addition to, or as an alternative to, determining whether algorithms are associated with related text based on the associated text that is stored with each algorithm. For example, if a rule runs on blood pressure data it may be implicitly associated with blood pressure.

If no stored algorithms are associated with a related term, the processing of stage 40 ends at stage 42.

If at least one stored algorithm is associated with a related term, the processing of stage proceeds to stage 44. At stage 44, the rules circuitry 24 executes the stored algorithm that was found at stage 40. If multiple stored algorithms are associated with the related term, the rules circuitry 24 executes all of the multiple stored algorithms. Each stored algorithm is applied to a structured data type that is specified in, or associated with, the stored algorithm. For example, a stored algorithm that finds instances of high blood pressure is defined to run on structured blood pressure data.

In other embodiments, the rules circuitry determines whether any actions are associated with the related term. The actions may comprise any suitable rules or algorithms that are performed on structured data, for example on laboratory data, vital signs data, medication data, observation data, or imaging data. In one example, an action comprises automated image processing, for example looking for calcium buildup. An action may comprise any suitable piece of automation. Examples of rules include:
“which blood pressure values have a systolic value >140 and diastolic value >90”
“which lab blood glucose values are >11.0 mmol/L”
“which prescribed medication names match ‘Simvastatin’”
“which DICOM series are of type MG?”
“is the patient female and between ages 18-65 and which patient weight values are below 50 kg?”

Examples of other pre-defined algorithms include image analysis algorithms, for example to identify a location of a DICOM series based on image contents and to act on any DICOM series where the location is related to the search term.

At stage 46, for each stored algorithm, the rules circuitry 24 determines whether running the stored algorithm has returned any results. For example, in the case of an stored algorithm that finds instances of high blood pressure, the stored algorithm may only return results if instances of high blood pressure are present in the structured blood pressure data. In an example of an image analysis algorithm, a user has searched for ‘stroke’, the search circuitry 26 determined that stroke is related to ‘head’, the image analysis algorithm identifies from image data that a DICOM series is of a head, so the algorithm returns the result that there is a relevant head scan.

If no results of the stored algorithms are found at stage 46, the process of running the stored algorithms ends at stage 48.

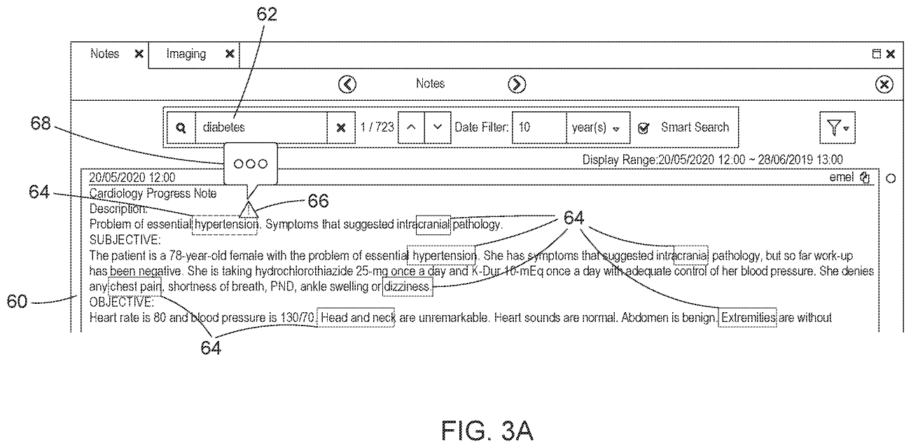

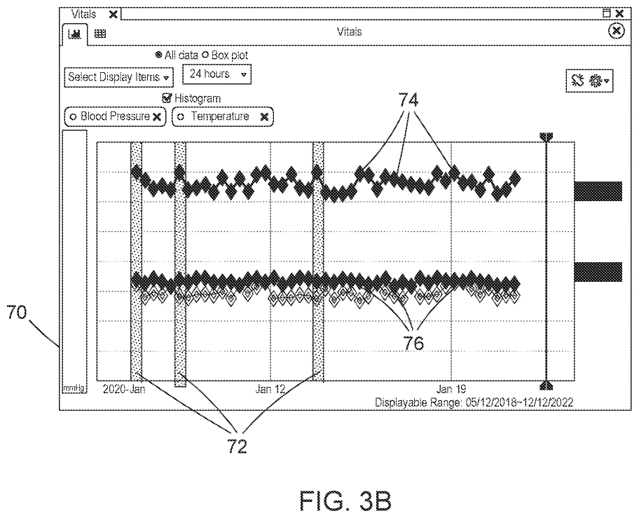

If one or more results are found at stage 46, the method of FIG. 2 proceeds to stage 50. At stage 50, the display circuitry 28 notifies the user that results have been found. Some examples of notification are described below with reference to FIGS. 3A to 6.

For example, the display circuitry 28 may display an icon as described with reference to FIG. 3A to indicate that results have been found. The user may interact with the icon, for example by clicking or mousing over the icon, to display results. Results may be summarised in a pop up, tool tip or other display. Summaries may be out of context summaries. Summaries may be displayed separately to the data that they summarize.

In some embodiments, if a user interacts with a notification icon then results are displayed and indicated in context. In some embodiments, if a user interacts with a result presented in a list, then the result is displayed and indicated in context. In some embodiments, if a user interacts with a result presented in a list, then a summary of the results is presented.

Results may be displayed on a panel, window or other display space that is configured to display structured data of the appropriate type. In some embodiments, the display circuitry 28 highlights data points or regions in a display of structured data displayed on display screen 16, for example on a plot of blood pressure data. In other embodiments, any suitable method of display may be used. For example, the display circuitry 28 may highlight items of structured information using a coloured background region, an outline, a change of font or style, a flag, or any other suitable highlighting method.

A panel showing results may be displayed adjacent to a panel that the user was using to search. In one example, the user searches for the term ‘high blood pressure’ and ‘Simvastatin’ is found to be a related term. ‘Simvastatin’ is known to represent the concept ‘medication’. A medication panel showing data relating to medication is shown on a panel adjacent to a panel that is being used to search, for example adjacent to a clinical notes panel.

In some embodiments, the presentation of the results to the user is discreet. In some embodiments, the presentation of the results to the user is more obvious.

At stage 52, the display circuitry 26 indicates to the user which related term initiated each of the stored algorithms for which results were obtained. The related term that initiated a stored algorithm is a related term that is associated with the stored algorithm and caused the stored algorithm to be selected at stage 40. The related term that initiated the algorithm may be the search term that was input at stage 30, or another related term of the related terms that were generated at stage 32.

In other embodiments, the display circuitry 26 may not indicate to the user which related term initiated each algorithm and stage 52 may be omitted.

In one example, the search term is ‘diabetes’. The related terms include ‘High Blood Pressure’. The stored algorithms that are executed include a stored algorithm that identifies instances of high blood pressure in the structured data. The display circuitry 28 indicates to the user that instances of high blood pressure have been identified as a result of ‘High Blood Pressure’ being a related term. Any suitable method of indicating the related term that initiated each stored algorithm may be used.

In another example, the search term is ‘stroke’ and the related terms include ‘head’. The stored algorithms that are executed include an image analysis algorithm that identifies DICOM series of the head. The display circuitry 28 indicates to the user that DICOM series have been identified as a result of ‘head’ being a related term.

Once the related terms have been indicated to the user, the process stops at stage 54.

In other embodiments, stages of FIG. 2 may be performed in any appropriate order. For example, instances of the related terms may be identified in the search before or after associated actions (for example, rules) are identified by the rules circuitry 24.

In summary, a user or another person associates terms with automation. In the embodiment of FIG. 2, the automation comprises a plurality of algorithms, which may also be described as rules. When the user performs a search then the text-expanding semantic search function identifies words and terms related to a search term. The rules circuitry 24 automatically runs any automation associated with related terms, which may include the search term. The display circuitry 28 presents the results to the user.

In the embodiment of FIG. 2, the rules circuitry 24 runs all algorithms that are associated with any of the related terms at stage 44. The rules circuitry 24 does not take into account whether instances of the related terms have been found in the unstructured text data at stage 36.

In other embodiments, the results of the search at stage 36 are used to refine the plurality of related terms that were generated by the search circuitry at stage 32. The search circuitry determines a set of further terms that include only the related terms for which instances were found to be present in the unstructured text data according to the results of the search at stage 36. At stage 40, the rules circuitry 24 identifies algorithms that are associated with the further terms.

The rules circuitry 24 only runs algorithms associated with a related term if at least one instance of that related term was found in the unstructured text data at stage 36. The rules circuitry 24 only runs automation associated with terms that are found in the patient record. In contrast, in the method of FIG. 2, the further terms used in finding associated algorithms are the same as the related terms generated at stage 32. In some circumstances, it may be desirable to limit the running of a rule such that it is only run if it relates to more than one search term, for example if there is a concern that the rule may otherwise be run too often. In some embodiments, an action is performed as soon as it is identified that a related term is associated with the action. In other embodiments, an action is performed once an instance of the related term has been found in the patient record.

In some embodiments, the rules circuitry 24 determines whether any of the stored algorithms are associated with the search term that was input by the user as a user input. The rules circuitry 24 determines, for each of the related terms, whether any of the stored algorithms are associated with the related term. The rules circuitry 24 executes any algorithm that is associated with the search term and is also associated with at least one of the related terms. The rules circuitry 24 may impose a condition that a rule is only executed if it is associated with both the search term and a related term. In other embodiments, any suitable action may be performed in response to a determination that the action is associated with both the search term and a related term.

In other embodiments, the rules circuitry 24 selects or modifies an algorithm or other action to be executed based on at least one item of patient information. The at least one item of patient information is obtained from the set of medical data for the patient. For example, the at least one item of patient information may be obtained from structured data in the set of medical data and/or by processing of unstructured text data in the set of medical data. The at least one item of patient information may comprise demographic information, for example patient age or gender. The at least one item of patient information may comprise information about a patient's disease status, for example whether the patient has diabetes. The at least one item of patient information may comprise information obtained from structured data such as lab data or vital signs data.

In some embodiments, the rules circuitry 24 determines, for each of the related terms, whether any of the stored algorithms are associated with the related term. If the rules circuitry 24 determines that at least one of the stored algorithms is associated with the related term, the rules circuitry 24 uses the at least one item of patient information to determine whether or how the algorithm is to be executed. For example, in one embodiment, the algorithm may be executed only if the patient is over a predetermined age.

In another embodiment, a high blood pressure algorithm is applied using different threshold values in dependence on patient information. A threshold value is set in dependence on the patient's age and diabetes status. For diabetic patients, the threshold value is set at 130/80 mmHg for blood pressure in the examining room and at 127/75 for blood pressure at home. For young, middle-aged and elderly patients, the threshold value is set at 140/90 mmHg for blood pressure in the examining room and at 135/85 for blood pressure at home. For late elderly patients, the threshold value is set at 150/90 mmHg for blood pressure in the examining room and at 145/85 for blood pressure at home. The rules circuitry 24 runs the high blood pressure algorithm using the threshold value that was set using the patient's age and diabetes status. The high blood pressure algorithm is run on structured blood pressure data for the patient. For a datum when the blood pressure is greater than the threshold value, the rule results in a pass. For a datum when the blood pressure is less than the threshold value, the rule results in a fail.

By using patient information in addition to the related terms when deciding whether or how to run an algorithm or perform another action, more relevant information may be displayed to a clinician.

In further embodiments, the rules circuitry 24 considers the search term, the related term and the at least one item of patient information when determining whether to execute an algorithm or perform another action. For example, in some embodiments, the rules circuitry 24 may only execute an algorithm if the algorithm is associated with the search term and the algorithm is associated with at least one related term and a condition is met by the at least one item of patient information. For example, the condition may be that the patient is a specified gender or that the patient exceeds a predetermined age threshold. In other embodiments, the rules circuitry 24 may always execute an algorithm if the associated with both the search term and a related term, but how the algorithm is executed may depend on the at least one item of patient information. For example, at least one threshold value used in the algorithm may be dependent on the at least one item of patient information.

The system of FIG. 1 performing the embodiment of FIG. 2 or related embodiments may automatically find and present key information to a user at the right time.

It may be considered that the user is providing an implicit clinical context through their search term.

In some cases, it may be expected that a user will want to perform a particular next action after a given search or after certain findings. By using the user's implicit clinical context, the user's next action may be pre-empted. Automation may be used to provide useful information to the user without the user requesting the information explicitly. For example, one or more rules may be run without the user having to actively decide to run the one or more rules.

The use of implicit clinical context may mean the system is less likely to waste clinician time with notification of things that they are not interested in. The use of implicit clinical context may mean the system is less likely to waste hardware resources running algorithms too speculatively.

Relevant information may be provided to the user in a way that is easy for the user to interpret. The use of relevant terms and of algorithms associated with the related terms may be transparent to the user. The user may understand why each algorithm has been run.

FIGS. 3A and 3B are representative of screen displays for an embodiment in which high blood pressure is found and indicated in structured data that forms part of a set of medical data associated with a patient. The set of medical data further comprises a set of clinical notes for the patient. FIG. 3A illustrates a clinical notes panel 60. FIG. 3B illustrates a vital signs panel 70.

In the embodiment of FIGS. 3A and 3B, the user inputs the search term ‘diabetes’ to search for the term ‘diabetes’ in the set of clinical notes. FIG. 3A shows the search term ‘diabetes’ when it is input into a search box 62.

The search circuitry 26 uses the text-expanding semantic search function to identify various terms as related to ‘diabetes’.

The related terms include ‘Hypertension’, ‘High blood pressure’ and ‘High BP’. The related terms also include ‘cranial’, ‘chest pain’, ‘dizziness’, ‘head and neck’ and ‘extremities’.

The search circuitry 26 then finds any instances of any of the related terms in the clinical notes. At least part of the set of clinical notes is displayed on the clinical notes panel 60. For example, part of the set of clinical notes may be displayed and the remainder of the clinical notes may be visible by scrolling.

The search circuitry 26 returns a list of search findings 64 comprising all the instances of the related terms that have been found in the clinical notes. The display circuitry 28 highlights the search findings 64 in the clinical notes panel 60. For example, the search identifies findings that relate to ‘diabetes’ and so the term ‘hypertension’ is indicated as a search finding.

The rules circuitry 24 identifies whether any of the related terms has an associated rule. In the example shown in FIG. 3A, the rules circuitry 24 identifies that there is a rule associated with ‘hypertension’. ‘Hypertension’ is one of the related terms and is present in the clinical notes.

The rules circuitry 24 identifies the association between the rule and the related term ‘hypertension’ and runs the rule. The rule searches a set of structured blood pressure data for blood pressure that is high, for example 140/90. At least part of the available structured blood pressure data for the patient is shown on the vital signs panel 70 as illustrated in FIG. 3B. The rule identifies regions for which blood pressure is high, where the regions are intervals of time.

The display circuitry 28 displays an icon 66 next to the term ‘hypertension’ in the clinical notes panel 60. The icon 66 notifies the user that results of a rule relating to the term ‘hypertension’ are available. In the embodiment of FIG. 3A, a summary of results of the rule is displayed in a pop-up text box 68 beside the icon 66.

The user may click on the icon 66 to display findings that were obtained by running the rule. In the embodiment of FIG. 3A, clicking on the icon 66 takes the user to a vital signs panel 70 as shown in FIG. 3B. In other embodiments, clicking on the icon 66 may display results of the rule in any suitable manner, for example as pop-up text comprising summary information.

Results may be unobtrusively presented to the user. The user may easily ask for more information or jump to the relevant panel and place. For example, clicking on the icon 66 may cause the vital signs panel 70 to be displayed on which regions of high blood pressure are highlighted. Alternatively, the user may choose to view blood pressure on a vital signs panel 70, for example by clicking on a different tab from the tab for the clinical notes panel 60 and by selecting blood pressure for display.

An example of a vital signs panel 70 is illustrated in FIG. 3B. The vital signs panel 70 shows a plurality of systolic blood pressure data points 74 and diastolic blood pressure data points. The rule referred to above in relation to FIG. 3A is run on blood pressure data comprising the systolic blood pressure data points and diastolic blood pressure data points that are represented in the vital signs panel 70.

The user may choose to display blood pressure on the vital signs panel 70 after ‘hypertension’ has been identified as a related term and the rule associated with ‘hypertension’ has been run on the structured blood pressure data. The display circuitry 28 highlights regions 72 in which high blood pressure is present in the vital signs panel. In the embodiment of FIG. 3B, the regions 72 of high blood pressure are identified using a coloured background. In other embodiments, any suitable method of distinguishing the regions of high blood pressure may be used. In further embodiments, any suitable method may be used to highlight the results of any suitable rule.

By displaying an icon beside ‘hypertension’ on the clinical notes panel to notify the user that a rule associated with ‘hypertension’ has been run, the user may easily see that further relevant data is available, and may navigate to see the identified regions of high blood pressure.

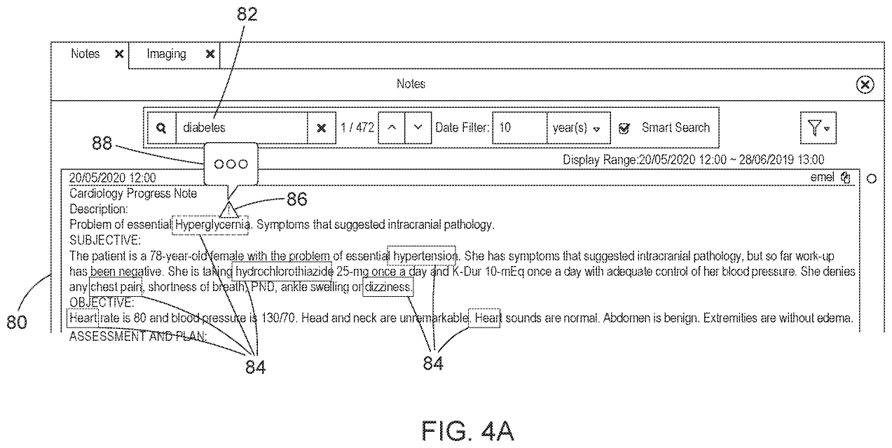

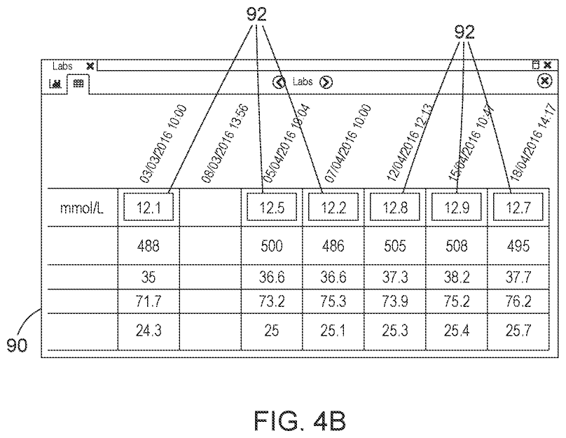

FIGS. 4A and 4B are representative of screen displays for an embodiment in which high glucose level is found and indicated in structured data. The structured data forms part of a set of medical data associated with a patient. The set of medical data also comprises clinical notes. FIG. 4A illustrates a clinical notes panel 80. FIG. 4B illustrates a lab data panel 90.

The user searches for ‘diabetes’ in the clinical notes by typing ‘diabetes’ into a search box 82. The text-expanding semantic search function identifies various words and terms as related, including ‘Hyperglycaemia’. Related terms also include ‘hypertension’, ‘hydrochlorothiazide’, ‘chest pain’, ‘dizziness’ and ‘heart’. The text-expanding semantic search function finds instances 84 of the related terms in the clinical notes and highlights related terms in the clinical notes panel 80 using any suitable method of highlighting.

The rules circuitry 24 determines whether there are any rules associated with the related terms that were identified by the text-expanding semantic search function. In the example shown in FIG. 4A, there is a rule associated with ‘Hyperglycaemia’ that will look for high blood glucose levels e.g. glucose >11.0 mmol/L. The rules circuitry 24 identifies the association and runs the rule associated with ‘Hyperglycaemia’ on a set of structured lab data comprising glucose data. The rule identifies high blood glucose levels.

The results are unobtrusively presented to the user using an icon 86 and pop up 88 as described above with relation to FIG. 3A. The user can easily ask for more information or jump to the relevant panel and place. Clicking on the icon 86 may take the user to a lab data panel 90 as described below with reference to FIG. 4B. In other embodiments, any suitable method of display may be used and the user may interact with the icon 86 in any suitable way.

FIG. 4B shows a lab data panel 90 which shows glucose values amongst other lab values. The rule associated with ‘Hyperglycaemia’ has been applied to the glucose values shown in the lab data panel 90. The rules circuitry 24 has highlighted in the lab data panel a plurality of high glucose values 92 as identified by the rule associated with ‘Hyperglycaemia’. In the embodiment of FIG. 4B, high glucose values 92 are highlighted by using a coloured background behind each data value that is identified by the rule. In other embodiments, any suitable method of highlighting may be used.

In a further embodiment, the user inputs the term ‘High cholesterol’ to search a set of clinical notes associated with a patient. The search circuitry 26 uses the text-expanding semantic search function to identify various words and terms as related to ‘High cholesterol’, including ‘Simvastatin’. The text-expanding semantic search function also identifies and returns to the system that information that ‘Simvastatin’ is a ‘medication’, through use of medical ontology.

The rules circuitry 24 identifies some automation, which may comprise a rule, that is linked to the concept of ‘medication’. The rules circuitry 24 runs the automation. The automation examines a set of structured medication data associated with the patient for the medication ‘Simvastatin’.

If there are any results from the search for ‘Simvastatin’ in the structured medication data, then this is unobtrusively indicated to the user, and the user can easily ask for more information or jump to the relevant panel and place. For example, clicking on an icon may allow the user to jump to a medication panel. The medication panel may provide information on when Simvastatin has been given, and how much Simvastatin has been given.

In a further embodiment, the user inputs the search term ‘Fibroadenoma’ to search a set of clinical notes associated with a patient. The set of clinical notes forms part of a set of medical data which also comprises a plurality of medical image data sets. In this embodiment, the medical image data sets are DICOM data sets. The medical image data sets may also be described as scans. The medical image data sets may comprise data obtained using any suitable medical imaging modality.

The search circuitry 26 uses the text-expanding semantic search function to identify various words and terms as related to ‘Fibroadenoma’, including ‘mammography’ and ‘mama’.

The rules circuitry 24 identifies some automation, for example a rule, that is linked to the terms ‘mammography’ and ‘mama’. The rules circuitry 24 runs the automation. The automation examines the DICOM description fields and protocols for the medical imaging data sets and finds any scans that comprise the terms ‘mammography’ or ‘mamo’.

If there are any results then this is unobtrusively indicated to the user. The user can easily ask for more information or jump to the relevant panel and place. For example, an icon may be displayed beside the term ‘mammography’ in the clinical notes. Clicking on the icon may open an imaging carousel, which may filter to show the ‘mamo’ scans.

In some circumstances, a related term is identified using the text-expanding semantic search function but is not present in the clinical notes. In some embodiments, the rules circuitry 24 identifies and runs rules that are associated with any of the related terms that are identified, even if the related term is not present in the clinical notes.

In such embodiments, it may not be possible to provide a notification against a finding within the clinical notes, where the finding is an instance of a related term. Instead, the notification may be placed elsewhere on the clinical notes panel.

In some embodiments, an icon is placed against the search term. Clicking on the icon may take the user to results of the rule.

In one embodiment, warfarin is identified as a term that is related to the search term, but is not present in the clinical notes. A pop-up text box beside the search term displays the text:

“The rule anticoagulant medication has findings related to the unseen term warfarin

| | |
|---|---|
| 28.11.20 | 6 mg |
| 14.10.20 | 7 mg” |

In another embodiment, multiple rules are identified that are associated with one or more related terms that are not present in the clinical notes. A pop-up text box beside the search term displays the text:
“There are multiple unseen findings. Please click the icon to display a list of these findings.”

In other embodiments, any suitable method may be used to notify the user of the results of Rules that are associated with related terms where the related terms are not present in the clinical notes.

The display circuitry 28 may display a list of findings to the user, where the findings are results of one or more rules. For example, a list of findings may be displayed on a findings panel.

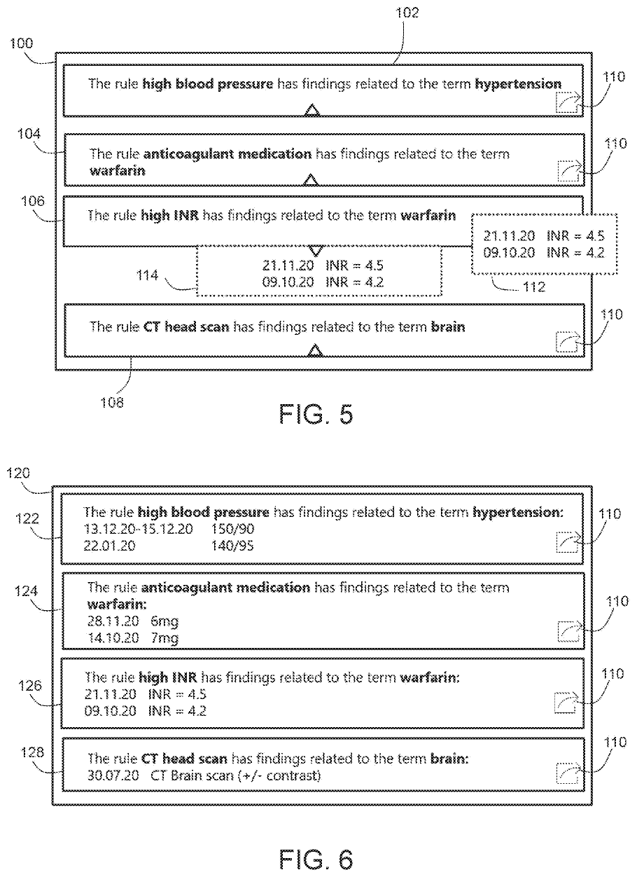

FIG. 5 illustrates an example of a findings panel 100 in which notifications 102, 104, 106, 108 are displayed as a list of findings. In the embodiment of FIG. 5, the user searches for ‘Stroke’. The search circuitry 26 uses the text-expanding semantic search function to identify related terms for the search term ‘Stroke’. Some related terms are present in a set of clinical notes.

In the embodiment of FIG. 5, both seen and unseen findings are presented in the list on the findings panel 100. In other embodiments, only unseen findings, or only seen findings, may be presented on the list.

A first notification 102 comprises the text ‘The rule high blood pressure has findings related to the term hypertension’.

An arrow symbol 110 is provided as part of the first notification 102. Clicking on the arrow symbol allows a user to launch a vital signs panel (not shown in FIG. 5) directly from the first notification 102.

A second notification 104 comprises the text ‘The rule anticoagulant medication has findings related to the term warfarin’. An arrow symbol 110 is provided as part of the second notification. Clicking on the arrow symbol 110 allows a user to launch a medication panel (not shown in FIG. 5) directly from the second notification 104.

A third notification 106 comprises the text ‘The rule high INR has findings related to the term warfarin’, where INR stands for International Normalized Ratio. An arrow symbol 110 is provided as part of the third notification. Clicking on the arrow symbol 110 allows a user to launch a lab data panel (not shown in FIG. 5) directly from the third notification 106.

A fourth notification 108 comprises the text ‘The rule CT head scan has findings related to the term brain’. An arrow symbol 110 is provided as part of the fourth notification. Clicking on the arrow symbol 110 allows a user to launch an imaging panel (not shown in FIG. 5) directly from the fourth notification 108.

FIG. 5 also shows examples of how further information may be provided to a user. A pop up summary 112 is displayed when the user hovers over the third notification 106. The pop up summary 112 includes brief information related to the third notification 106, which comprises the text:

| | |
|---|---|
| ‘21.11.20 | INR = 4.5 |
| 09.10.20 | INR = 4.2’ |

An expanded summary 114 may be obtained by clicking on the third notification 106 to expand the listing for the third notification 106. In the embodiment shown, the same text is included in the expanded summary as in the pop-up summary. In other embodiments, different text may be included. In some embodiments, both a pop-up summary and an expanded summary are available for each of the notifications. In other embodiments, only pop-up summaries are available or only expanded summaries are available. In further embodiments, any suitable method of providing additional information for a notification may be used. In some embodiments, results of a text search may be displayed in combination with automation results.

FIG. 6 illustrates a list of findings provided on a findings panel 120 according to an embodiment. In the embodiment of FIG. 6, a summary is provided as part of the list of findings. The user searches for ‘Stroke’. Seen and unseen findings are presented as a list of summaries.

A first notification 122 comprises the text ‘The rule high blood pressure has findings related to the term hypertension’ and the summary:

| | |
|---|---|
| ‘13.12.20-15.12.20 | 150/90 |
| 22.01.20 | 140/95’ |

An arrow symbol 110 is provided as part of the first notification 122. Clicking on the arrow symbol allows a user to launch a vital signs panel (not shown in FIG. 6) directly from the first notification 122.

A second notification 124 comprises the text ‘The rule anticoagulant medication has findings related to the term warfarin’ and the summary:

| | |
|---|---|
| ‘28.11.20 | 6 mg |
| 14.10.20 | 7 mg’ |

An arrow symbol 110 is provided as part of the second notification 124. Clicking on the arrow symbol 110 allows a user to launch a medication panel (not shown in FIG. 6) directly from the second notification 124.

A third notification 126 comprises the text ‘The rule high INR has findings related to the term warfarin’ and the summary:

| | |
|---|---|
| ‘21.11.20 | INR = 4.5 |
| 09.10.20 | INR = 4.2’ |

An arrow symbol 110 is provided as part of the third notification 126. Clicking on the arrow symbol 110 allows a user to launch a lab data panel (not shown in FIG. 5) directly from the third notification 126.

A fourth notification 128 comprises the text ‘The rule CT head scan has findings related to the term brain’ and the summary:

| | |
|---|---|
| ‘30.07.20 | CT Brain scan (+/−contrast)’ |

An arrow symbol 110 is provided as part of the fourth notification 128. Clicking on the arrow symbol 110 allows a user to launch an imaging panel (not shown in FIG. 5) directly from the fourth notification 128.

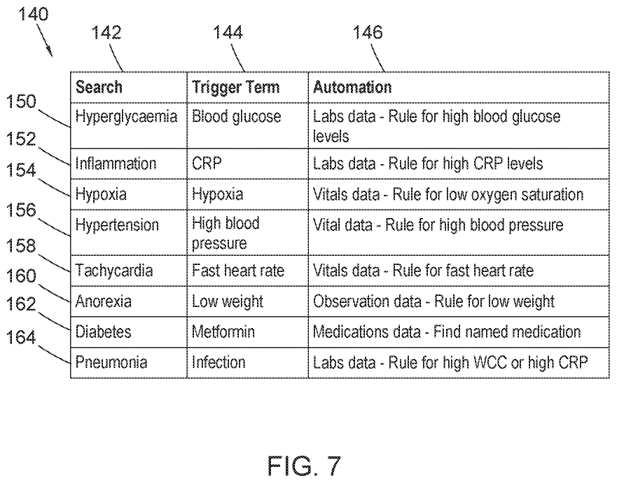

FIG. 7 shows a table 140 of examples of automation. A first column 142 comprises examples of terms that are searched. A second column 144 comprises examples of related terms that triggered an action. The related terms may also be referred to as trigger terms. A third column 146 comprises examples of the action performed.

In a first example 150, the search term that is input by a user is ‘Hyperglycaemia’. The search circuitry 26 identifies related terms including trigger term ‘blood glucose’. The rules circuitry 26 finds that ‘blood glucose’ is associated with automation comprising a rule for high blood glucose levels that is to be performed on lab data.

In a second example 152, the search term is ‘inflammation’ and the related trigger term is ‘CRP’. The rules circuitry 26 finds that ‘CRP’ is associated with automation comprising a rule for high CRP (C-Reactive Protein) levels that is to be performed on lab data.

In a third example 154, the search term is ‘hypoxia’ and the related trigger term is hypoxia’. The rules circuitry 26 finds that ‘hypoxia’ is associated with automation comprising a rule for low oxygen saturation that is to be performed on vital signs data.

In a fourth example 156, the search term is ‘hypertension’ and the related trigger term is ‘high blood pressure’. The rules circuitry 26 finds that ‘high blood pressure’ is associated with automation comprising a rule for high blood pressure that is to be performed on vital signs data.

In a fifth example 158, the search term is ‘tachycardia’ and the related trigger term is ‘fast heart rate’. The rules circuitry 26 finds that ‘fast heart rate’ is associated with automation comprising a rule for fast heart rate that is to be performed on vital signs data.

In a sixth example 160, the search term is ‘anorexia’ and the related trigger term is low weight’. The rules circuitry 26 finds that low weight’ is associated with automation comprising a rule for low weight that is to be performed on observation data.

In a seventh example 162, the search term is ‘diabetes’ and the related trigger term is ‘metformin’. The rules circuitry 26 finds that the ‘metformin’ is a medication and is associated with automation comprising a rule for finding a named medication, that is to be performed on medications data.

In an eighth example 164, the search term is ‘pneumonia’ and the related trigger term is ‘infection’. The rules circuitry 26 finds that ‘infection’ is associated with automation comprising a rule for high WCC (white cell count) or high CRP that is to be performed on labs data.

In embodiments described above, for example the embodiment of FIG. 2, a single search is performed by the user. The user inputs a search term, the search circuitry 26 determines a plurality of related terms, and the rules circuitry 24 finds and runs rules that are associated with the related terms. Optionally, the search circuitry 26 may refine the plurality of related terms to include only terms that were found in a search of text data, for example a set of clinical notes.

In further embodiments, the user inputs a first search term. The search circuitry 26 uses the text-expanding semantic search functionality to obtain a first set of related terms that are related to the first search term.

The user then inputs a second, subsequent search term. The search circuitry 26 uses the text-expanding semantic search functionality to obtain a second set of related terms that are related to the second search term. Some terms may occur in both the first set of related terms and the second set of related terms.

The search circuitry 26 selects related terms that are present in both the first set of related terms and the second set of related terms. The rules circuitry 24 runs any automation that is associated with the selected terms, which may also be described as further terms. The rules circuitry 24 may also indicate to the user which of the selected terms initiated the automation.

Optionally, the search circuitry 26 may search the clinical notes associated with the patient for instances of the related terms and may select only related terms that are present in all of: the first set of related terms, the second set of related terms, and the clinical notes.

Results may be presented to the user, either discretely or obviously. The user may provide an implicit clinical context through their use of both the first search term and the second search term.

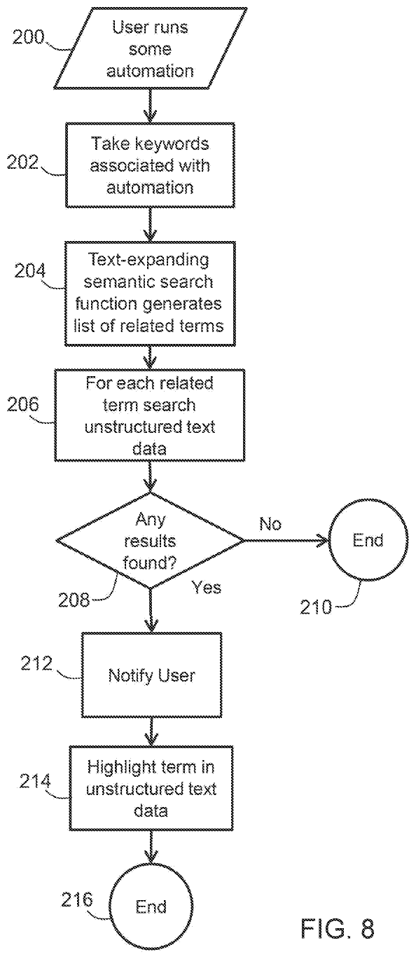

FIG. 8 is a flow chart illustrating in overview a method of an embodiment. The apparatus of FIG. 1 is configured to perform the method of FIG. 8. FIG. 8 is performed in relation to a set of medical data for a subject, which includes structured data and unstructured text data.

At stage 200 of FIG. 8, the user selects some automation and runs the automation. It may be considered that the user runs automation manually.

The user may select an action from a stored set of actions. For example, the user may select a rule from a drop-down list of rules. Each action may be performed on structured data, for example laboratory data, vital signs data, observation data, medication data, or imaging data.

The rules circuitry 26 receives a user input, for example a drop-down selection made by a user. The user input instructs the performance of an action on a portion of the set of medical data, which may be referred to as first medical data.

In one example, a rule called High BP is selected by the user. By selecting the High BP rule, the user instructs the High BP rule to be executed on the first medical data. The rules circuitry 24 executes the rule and outputs any results. In the case of the High BP rule, the rule is run on vital signs data and the results may comprise regions of high blood pressure. Results may be displayed by highlighting regions of high blood pressure within a vital signs panel.

Each action, for example each rule, has associated text which is entered by the user when the rule is created. For example, for the High BP rule, the associated text includes the terms ‘Hypertension’ and ‘High blood pressure’. The associated text comprises one or more clinical terms, which may also be described as keywords.

The rules circuitry 24 identifies at least one input term, where the at least one input term comprises any clinical terms associated with the selected action. Each input term may comprise a word, a partial word, or a group of words.

In other embodiments, the rules circuitry 24 may determine the at least one input term from the action in any suitable manner. For example, the at least one input terms may be determined based on a type of data that the action operates on.

At stage 202, the rules circuitry 24 passes the at least one input term to the search circuitry 26.

At stage 204, the search circuitry 26 uses the text-expanding semantic search functionality to generate a list of related terms for the at least one input term. Each related term is conceptually related to one or more keywords in the associated text. The related terms may be found using a clinical coding system or any suitable method. The related terms may also be referred to as further terms.

At stage 206, for each related term, the search circuitry 26 searches the unstructured text data for instances of the related term. For example, the unstructured text data may be clinical notes.

At stage 208, for each related term, the search circuitry 26 asks whether any search results have been found, where the search results are instances of the related term.

If no search results for the related terms have been found, the method of FIG. 8 ends at stage 210.

If one or more search results has been found, the method of FIG. 8 proceeds to stage 212.

At stage 212, the search circuitry 26 notifies the user that one or more search results have been found. In some embodiments, the user is notified using an icon within the context of the rule that has been selected by the user. For example, if the user selects the rule from a drop-down menu, the user may be notified by an icon on or beside the drop down menu. In other embodiments, the user may be notified by an icon within a display of results of the rule, for example an icon within a vital signs panel displaying results of the rule. In further embodiments, any suitable method of notification may be used to notify the user that search results are available.

The icon may be unobtrusively presented to the user, and the user may easily ask for more information or jump to findings in the notes panel, for example by clicking on the icon.

At stage 214, the search circuitry 26 highlights the search results in a clinical notes panel. The display circuitry 28 may highlight each instance of a related term in the set of clinical notes using a coloured background region, an outline, a change of font or style, a flag, or any other suitable highlighting method.

In one example in which the rule is a High BP rule, the search circuitry 26 presents and highlights the related term ‘hypertension’ in a clinical notes panel using a coloured background.

Once the search results have been displayed, the method of FIG. 8 ends at stage 216.

In the embodiment of FIG. 8, the user may be considered to be providing an implicit clinical context through the automation that they run. In some cases, it may be expected that a user will want to perform a particular search after running a rule or other action. By using the user's implicit clinical context, the user's next action may be pre-empted. Useful information may be provided to the user without the user requesting the information explicitly.

In the embodiment of FIG. 8, the user selects an action, one or more input terms associated with the action are identified, and the search circuitry 26 identifies related terms and performs a search for the related terms. In other embodiments, the user selects an action, one or more input terms associated with the action are identified, and the search circuitry 26 identifies related terms. The rules circuitry 24 then identifies and runs one or more further actions from a set of stored actions, for example as described above in relation to stages 40 to 54 of FIG. 2.

In other embodiments, features of any embodiments described above may be provided in any suitable combination. For example, any suitable features of the embodiment of FIG. 2 may be combined with any suitable feature of the embodiment of FIG. 8.

Certain embodiments provide a system comprising:
a. Access to a corpus of electronic medical data, some of which is represented as text;
b. A mechanism for the user to search that text data by providing a one or set of characters, this is the search term;
c. One or more actions that the system can automatically perform, that are potentially useful to the user, potentially depending on the context;
d. A heuristic that when provided with the search term can generate one or more new set of characters that are clinically relevant to the initial search term;
e. These newly generated set of characters may also be known to represent a clinical ‘concept’ such as ‘medication’, ‘body part’, etc;
f. in which:
   i. The user, or other, has associated one or more actions with one or more set of characters;
   ii. The user performs a search of the electronic medical data by providing one a search term;
   iii. The system uses the heuristic to generate one or more set of characters based on the user search term;
   iv. The system automatically checks to see if any action is associated with any of the set of characters generated by the heuristic, and if so, then automatically runs the action, or optionally only runs the action if the set of characters is also found in the text portion of the medical data.

Examples of automation may include, but are not limited to executing ‘rules’ that the user, or other, has defined. A ‘rule’ can examine structured data and perform logical operations, mathematical operations, and text operations and for each piece of structured data report if the rule ‘passes’.

Examples of rules include, but are not limited to:
a. “which blood pressure values have a systolic value >140 and diastolic value >90”
b. “which lab blood glucose values are >11.0 mmol/L”
c. “which prescribed medication names match ‘Simvastatin’”
d. “which DICOM series are of type MG?”
e. “is the patient female and between ages 18-65 and which patient weight values are below 50 kg?”

Examples of automation may include executing other pre-defined algorithms.

Examples of pre-defined algorithms may include, but are not limited to an Image Analysis algorithm to identify the location of the DICOM series based on the image contents and then acting on the ones where the location is related to the search term. User has searched for ‘stroke’, ‘stroke’ is related to ‘head’, the IA algorithm identifies from the image data that a DICOM series is of a head, so indicate to the user that there is a relevant head scan.

Examples of automation include, but are not limited to displaying for the user a ‘panel’ that shows the type of data relevant to the search term. Examples include, but are not limited to:
User has searched for ‘high blood pressure’, ‘Simvastatin’ is known to represent the concept ‘medication’, the ‘Medication panel’ is known to show data of the concept ‘medication’, and so the panel is shown to the user adjacent to the panel that the user was using to search.

If the system identified an associated action, and that associated action has been automatically run, and only if that action has resulted in any findings, then a notification may be displayed to the user.

A notification may comprise an icon displayed unobtrusively next to the search input.

The set of characters resulting from the heuristic may be found in the text portion of the electronic medical data, then a notification may comprise an icon displayed next to the found set of characters.

The notification may comprise a list of findings displayed to the user.

The notification may comprise a list of findings displayed as out of context summaries displayed to the user.

There may be finding(s), and the user may interact with the notification icon then the system may display and indicate the finding(s) in context.

There may be finding(s), and the user may interact with a finding in the list then the system may display and indicate the finding in context.

There may be finding(s), and the user may interact with a finding in the list then the system may display a summary of that finding.

The set of characters that initiated the action may be indicated along with the finding.

Certain embodiments provide a system comprising:
a. Access to a corpus of electronic medical data, some of which is represented as text;
b. A mechanism for the user to see the text data;
c. One or more actions that the system can automatically perform;
d. A heuristic that when provided with the search term can generate one or more new set of characters that are clinically relevant to the initial search term;
e. in which:
   i. The user, or other, has associated one or more actions with one or more set of characters;
   ii. The user initiates an action that the system automatically performs, and that action is associated with one or more set of characters;
   iii. The system uses the heuristic to generate one or more set of characters based on the user search term;
   iv. The system searches the text data for the set of characters associated with that action and if any of those sets are found in the text then the user is notified and the matching terms are highlighted in the text data.

Certain embodiments provide a system comprising:
a. Access to a corpus of electronic medical data, some of which is represented as text;
b. A mechanism for the user to search that text data by providing a one or set of characters, this is the search term;
c. One or more actions that the system can automatically perform, that are potentially useful to the user, potentially depending on the context;
d. A heuristic that when provided with the search term can generate one or more new set of characters that are clinically relevant to the initial search term;
e. These newly generated set of characters may also be known to represent a clinical ‘concept’ such as ‘medication’, ‘body part’, etc.;
f. in which:
   i. The user, or other, has associated one or more actions with one or more set of characters;
   ii. The user performs a search of the electronic medical data by providing one a search term;
   iii. The user performs one or more subsequent searches of the electronic medical data by providing one a search term;
   iv. The system uses the heuristic to generate one or more set of characters based on each of the user search terms;
   v. The system automatically checks to see if any action is associated with any of the set of characters generated by the heuristic by more than one of the search terms, and if so, then automatically runs the action, or optionally only runs the action if the set of characters is also found in the text portion of the medical data.

Certain embodiments provide a method of using a clinical information system, comprising: receiving input from a user; determining at least one search term based on the user input, wherein at least one action is associated with certain search terms, each action comprising applying a rule or algorithm; determining whether any actions are associated with the determined search terms; performing searches using the determined search terms; if there are rules or algorithms associated with the determined search terms then applying the rules or algorithms to at least one data set; displaying output to the user, wherein the output comprises both results of the searches and results of applying the at least one rule or algorithm to said at least one data set.

The user input may comprise selection of a rule or algorithm, and the applying of the rules or algorithms may include applying at least the rule or algorithm selected by the user.

The user input may comprise a first search term, and the determining of at least one search term may comprise determining at least one search term related to the first search term.

The method may comprise performing the searches on electronic medical data, for example a set of electronic medical documents.

The rules or algorithms may be applied to stored medical images or other stored medical measurement data.

The displaying of output may comprise displaying a notification, optionally in response to an action resulting in any significant findings, and optionally:
a) the notification comprises an icon displayed next to the input; and/or
b) if the search term is found in a text portion of the electronic medical data, then a notification is an icon displayed next to the found set of characters; and/or
c) wherein the notification comprises a list of findings; and/or
d) wherein the notification comprises a list of findings displayed as out of context summaries; and/or
e) wherein if there are significant finding(s), and the user interacts with the notification icon or a finding in the list then the method comprises display and indicating the finding(s) in context; and/or
f) wherein if there are significant finding(s), and the user interacts with a finding in the list then the method comprises displaying and indicating the finding in context; and/or
g) wherein if there are significant finding(s), and the user interacts with a finding in the list then the method comprises displaying a summary of that finding

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. A clinical information system, comprising processing circuitry configured to:
receive a user input from a user, wherein the user input instructs the performing of a first action on first medical data for a subject;
determine based on the user input and/or the first action at least one input term;
determine at least one further term that is conceptually related to the at least one input term;
select at least one stored action of a set of stored actions, wherein the selecting of the at least one stored action comprises determining that the at least one stored action is associated with the at least one further term;
perform said at least one stored action on second medical data for the subject; and
provide to the user a notification of said stored action.

2. A system according to claim 1, wherein the user input comprises a search term; the at least one input term comprises the search term; the first medical data comprises text data; and the first action comprises a search of the first medical data for the search term.

3. A system according to claim 1, wherein each stored action comprises a rule or algorithm.

4. A system according to claim 1, wherein the second medical data comprises at least one of vital signs data, laboratory data, observation data, medication data, imaging data.

5. A system according to claim 1, wherein the determining of the at least one further term comprises using a clinical coding system, terminology or ontology to identify terms that are conceptually related to the input term.

6. A system according to claim 5, wherein the first medical data comprises text data, and the processing circuitry is further configured to search the first medical data to identify instances of the terms that are conceptually related to the input term.

7. A system according to claim 6, wherein the determining of the at least one further term comprises excluding any term that is conceptually related to the input term but is not present in the text data.

8. A system according to claim 1, wherein at least one of a), b) and c): —
a) the selecting of the at least one stored action further comprises determining that the at least one stored action is associated with the input term;
b) the selecting of the at least one stored action is in dependence on at least one item of patient information obtained from the first medical data;
c) the selecting of the at least one stored action is in dependence on at least one item of patient information obtained from the second medical data.

9. A system according to claim 1 wherein the notification comprises at least one:
a) displaying an icon near to the user input;
b) displaying an icon near to an instance of a further term in the first medical data;
c) displaying a summary of results of the at least one stored action;
d) displaying a list of results of the at least one stored action.

10. A system according to claim 1, wherein the processing circuitry is further configured to display at least part of the second medical data and to highlight results of the at least one stored action in the second medical data.

11. A system according to claim 1, wherein the first medical data comprises text data, and the processing circuitry is further configured to display at least part of the first medical data and to highlight instances of the at least one further term in the first medical data.

12. A system according to claim 1, wherein, for each stored action, the notification of said stored action comprises a notification of which further term or terms are associated with said stored action.

13. A system according to claim 1, wherein the processing circuitry is further configured to receive a second user input and to determine based on the user input at least one second input term;
wherein the determining of the at least one further term comprises determining terms that are conceptually related to both the input term and the second input term.

14. A system according to claim 1, wherein the user input comprises a selection of a rule or algorithm, and the at least one input term comprises at least one term associated with the selected rule or algorithm.

15. A method comprising:
receiving a user input from a user, wherein the user input instructs the performing of a first action on first medical data for a subject;
determining based on the user input at least one input term;
determining at least one further term that is conceptually related to the at least one input term;
determining whether any stored action of a set of stored actions is associated with the at least one further term; and,
if a stored action is associated with the at least one further term,
performing said stored action on second medical data for the subject; and
providing to the user a notification of said stored action.

16. A clinical information system, comprising processing circuitry configured to:
receive a user input from a user, wherein the a user input instructs the processing circuitry to perform a first action on first medical data for a subject, wherein the first action comprises a rule or algorithm;
determine based on the user input at least one input term that is associated with the first action;
determine at least one further term that is conceptually related to the at least one input term;
determine whether instances of the at least one further term are present in second medical data for the subject, wherein the second medical data comprises text data; and
if instances of the at least one further term are present in the second medical data, provide to the user a notification of said instances.

17. A system according to claim 16, wherein the user input comprises a selection of a stored action from a set of stored actions.

18. A system according to claim 16, wherein the notification of said instances comprises displaying at least part of the second medical data and highlighting instances of the at least one further term in the first medical data.

19. A system according to claim 16, wherein the determining of the at least one further term comprises using a clinical coding system, terminology or ontology to identify terms that are conceptually related to the input term.

20. A system according to claim 16, wherein the first medical data comprises at least one of vital signs data, laboratory data, observation data, medication data, imaging data.

21. A system according to claim 1, wherein the processing circuitry is further configured to display at least part of the first medical data and to highlight results of the first action in the first medical data.

22. A method comprising:
receiving a user input from a user, wherein the user input instructs the processing circuitry to perform a first action on first medical data for a subject, wherein the first action comprises a rule or algorithm;
determining based on the user input at least one input term that is associated with the first action;
determining at least one further term that is conceptually related to the at least one input term;
determining whether instances of the at least one further term are present in second medical data for the subject, wherein the second medical data comprises text data; and,
if instances of the at least one further term are present in the second medical data, providing to the user a notification of said instances.
